(19) United States
(12) Patent Application Publication
(10) Pub. No.: US 2005/0004535 A1
(43) Pub. Date: Jan. 6, 2005

(54) Device for the removal of fluids during surgical procedures

(76) Inventor: Peter A. Schklair, Midlothian, TX (US)

Correspondence Address: Squire, Sanders & Dempsey L.L.P., Two Renaissance Square, 40 North Central Avenue, Suite 2700, Phoenix, AZ, 85004-4498, US

(21) Appl. No.: 10/428,456

(22) Filed: May 1, 2003

(51) Int. Cl. A61M 1/00

(52) U.S. Cl. 604/317

(57) ABSTRACT

A device for removing fluids during surgery may be positioned into or onto a surgical site by a surgeon or assistant. The device preferably includes two arms that can be positioned on either side of an incision, wherein the arms have apertures communicating with an internal cavity. Suction is applied to the internal cavity and fluid is drawn into the apertures, through the cavity and into a containment receptacle. The device preferably includes a handle that allows a surgeon to move the device and apply pressure as needed to the surgical site.

### BACKGROUND OF THE INVENTION

The present invention is directed to a device that may be connected to a hose having a suction source attached thereto for use during surgical procedures for the removal of fluids, e.g., blood from an operation site. More particularly, the present invention is directed to a device for use during a cesarean section procedure to ensure that the operating field is relatively free of blood while incising the lower uterine segment.

Suction devices for use during surgical procedures for the removal of fluids, e.g., blood, from an operation site, are, in general, known. Devices are typically adapted for connection to a suction source through a flexible hose. One type of prior art suction device is commonly known as a Yankauer. A Yankauer typically comprises a generally elongated tubular member having substantially constant inner and/or outer diameters. A tip is provided at one end of the tube for disposition into the operation site through which suction pick-up of fluids, tissue, and so forth, is effected. The other end of the tube is adapted for attachment to a hose, which is, in turn, connected to a suction source. Nipples or special fittings are often employed to provide secure attachment to the hose. The suction tube may also include a flared or widened portion along its length to facilitate grasping the tube. The suction tube is provided in various shapes, e.g., curved, to facilitate positioning of the tip.

Another type of prior art suction device is commonly known as a Poole suction instrument. A Poole instrument likewise typically comprises a generally elongated tubular member, but a plurality of apertures are included through the outer wall of the tubular member through which suction pickup of fluids is effected.

Examples of prior art suction devices are described in the following U.S. Pat. No. 3,623,483 issued to Dyer on Nov. 30, 1971; U.S. Pat. No. 5,358,507 issued to Daily on Oct. 25, 1994; U.S. Pat. No. 5,643,229 issued to Sinaiko on Jul. 1, 1997; U.S. Pat. No. 5,685,836 issued to DiPema, et al. on Nov. 11, 1997; U.S. Pat. No. 5,738,648 issued to Lands et al. on Apr. 14, 1998; U.S. Pat. No. 5,921,970 issued to Vandenberg on Jul. 13, 1999; and U.S. Pat. No. 6,086,587 issued to Hawk on Jul. 11, 2000.

The use of such suction devices is particularly important to prevent blood and other fluids from obscuring the surgeon's view. This is particularly true with respect to surgical procedures where extensive bleeding may occur. For example, in a cesarean section procedure, extensive bleeding is commonly encountered when incising the lower uterine section. However, is important that the surgeon have a clear view of the operating field to avoid potential injury to the underlying fetus.

### SUMMARY OF THE INVENTION

The present invention provides, in accordance with one aspect, an improved device for the removal of fluids from the vicinity of an incision made during a surgical procedure. This is accomplished by providing arms that, response to pressure, tend to compress the tissue and occlude the blood vessels on either side of the incision, while at the same time, responsive to application of suction, removing blood and fluids from the operating field through apertures in the sidewalls of at least one of the arms. Such a device preferably comprises a base including a first arm and a second arm and an interior cavity extending into each of the arms. The respective arms are disposed to extend along opposite sides of the nominal line along which the incision is to be made. At least one arm, and preferably both arms, includes a plurality of apertures communicating between the interior cavity in the arm and exterior of the arm facing the incision line. Provisions are made for the application of suction to the interior cavity.

In the preferred embodiment, the device further includes a handle (fixedly or removably attached) extending from the base at an angle chosen to facilitate application of force to the arms to compress the tissue and occlude the blood vessels on either side of the incision. In the preferred embodiment, suction is applied to the interior cavity through a conduit in the handle.

### BRIEF DESCRIPTION OF THE DRAWING

The preferred exemplary embodiment of the present invention will hereinafter be described in conjunction with the appended drawing, where like designations denote like elements, and:

FIG. 1 is a schematic diagram of the use of a suction device to remove fluids from an operating field;

FIG. 2 is a partially cut-away perspective view of a suction device in accordance with one embodiment of the present invention;

FIG. 3 is a perspective view (from below) of a suction device in accordance with another embodiment of the present invention.

FIG. 4 is a perspective view of a suction device in accordance with another embodiment of the present invention.

### DETAILED DESCRIPTION OF A PREFERRED EXEMPLARY EMBODIMENT

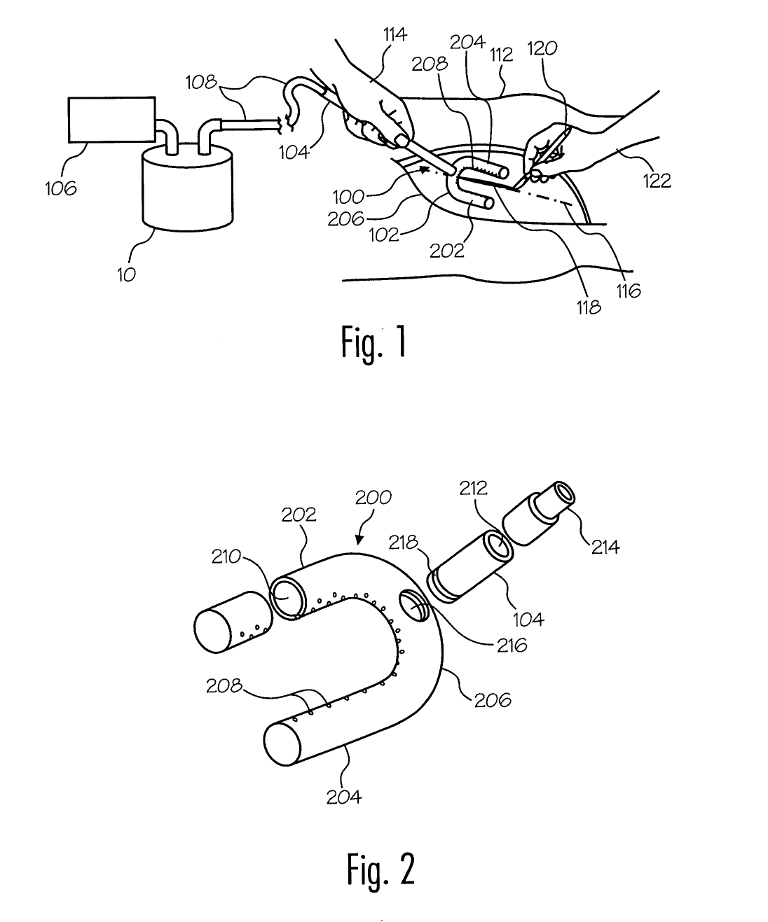

Referring to FIG. 1, a suction device 100 in accordance with the present invention suitably comprises a base 102, cooperating with a handle 104. In use, device 100 typically cooperates with a suitable suction system, e.g., one comprising a conventional regulated vacuum (suction) source 106, suction hose 108, and a waste receptacle 110. Vacuum source 106 conventionally provides suction at a predetermined (typically standardized by accreditation organizations) vacuum level, e.g., from 12 to 25 inches of mercury, to hose 108.

Device 100 is employed to remove fluids, e.g., blood, from an operation site 112, during a surgical procedure, and particularly procedures of the type where extensive bleeding may occur from an incision. For example, in a cesarean section procedure, extensive bleeding is commonly encountered when incising the lower uterine section. As will hereinafter be more fully described, base 102 includes respective spaced-apart arms (202, 204), at least one of which, and preferably both, including suction apertures (208) that ultimately communicate with vacuum source 106 and waste receptacle 110. A surgeon (or an assistant) grips handle 104 in one hand 114, inserts device 100 into or onto operation site 112, and preferably positions device 100 with arms 202 and 204 disposed on opposing sides of a nominal incision line 116 along which an incision 118 is intended to be made. The surgeon, with a tool, e.g., scalpel, 120 held in hand 122, makes an incision 118 between arms 202 and 204 along nominal line 116, using arms 202 and 204 as guides. At the same time, the surgeon (or assistant) exerts force on handle 104 to cause arms 202 and 204 to compress the underlying tissue and occlude the blood vessels in the vicinity of incision 118 to prevent excessive bleeding. Suction is also applied to apertures 208 so that blood and fluids from the operating field are drawn off through apertures 208, and ultimately through hose 108 and are deposited in waste receptacle 110.

More specifically, with reference now to FIGS. 1 and 2, in a first preferred embodiment, base 102 includes: a first arm 202; a second arm 204; a connecting portion 206; an interior cavity 210 (best seen in FIG. 2) extending through connecting portion 206 and into each of arms 202 and 204; and apertures 208, providing fluid communication between interior cavity 210 and the exterior of base 102. As noted above, apertures 208 are provided in at least one, and preferably both, of arms 202 and 204, and preferably in connecting portion 206 as well. In the embodiment of FIGS. 1 and 2, apertures 208 are disposed in the sidewalls of arms 202 and 204 (and connecting portion 206) and face nominal incision line 116 when device 100 is in use. As will further be explained, with device 100 in operation, cavity 210, and thus apertures 208, are ultimately connected to hose 108 and vacuum source 106. In the preferred embodiment, cavity 210 communicates with a passageway 212 running between the proximal and distal ends of handle 104; handle 104 is tubular and includes a nipple or other suitable fitting 214 at its distal end to receive hose 108. Thus, in such embodiment, the system providing suction to remove fluids comprises apertures 208, cavity 210, passageway 212, hose 108 and vacuum source 106.

The configuration (e.g., length, cross-section and relative disposition) of arms 202 and 204 is such that: the surgeon can make incision 118 with an unobstructed view; suction from apertures 208 removes fluids, and (although not required) pressure may be exerted on the underlying tissue to occlude the blood vessels in the vicinity of incision 118. To ensure that blood from incision 118 is sufficiently suctioned off through apertures 208, suction from apertures 208 should not be overly diluted; it is thus preferred, although not required, that the suction be maintained at least at a minimum value, e.g., at least 12 inches of mercury measured at sidewall of arms 202 and 204 (e.g., at apertures 208).

Arms 202 and 204 are spaced apart; e.g., are preferably (but not necessarily) parallel members having opposing sidewalls spaced apart by a distance D1. Relatively straight, parallel arms, while not essential, are advantageous in that uniform suction between the arms may be more easily facilitated, and straight arms provide a guide for the surgeon when making incision 118. In general, distance D1 is preferably chosen to be as wide as possible (so that the surgeon can make incision 118 with an unobstructed view), while still close enough to adequately remove the fluids from incision 118. Distance D1 is typically in the range of from 0.25 to 0.75 inches, and in the preferred embodiment, is approximately 0.5 inches.

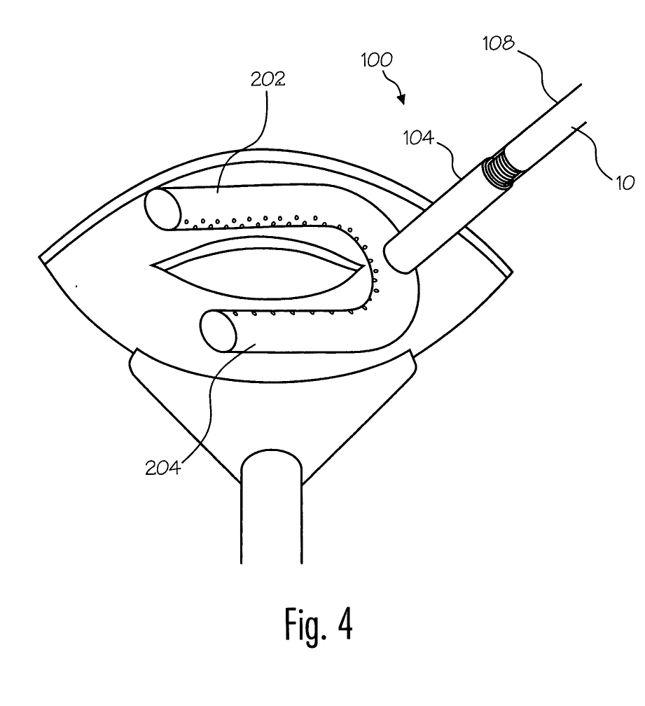

Arms 202 and 204 are preferably (but not necessarily) of identical cross-section, and sufficient to accommodate a cavity 210 capable of providing the requisite suction and fluid flow rates, while remaining strong enough to exert a desired pressure on underlying tissue. Cavity 208 is preferably (but not necessarily) concentric with, the exterior of arms 202 and 204 (and connecting portion 206). In the embodiment of FIG. 1, arms 202 and 204 (and connecting portion 206 ) are annular in cross-section with an exterior diameter ranging from 0.375 to 0.625 inches, preferably from 0.375 to 0.5 inches, and in the preferred embodiment approximately 0.375 inches, and an interior diameter (defining cavity 210) ranging from 0.125 to 0.5 inches, preferably from 0.25 to 0.375 inches, and in the preferred embodiment approximately 0.25 inches. The length D2 of arms 202 and 204 is such that with a given suction system (e.g., apertures 208, cavity 210, passageway 212, hose 108 and vacuum source 106) adequate suction will be provided through apertures 208. In the embodiment of FIGS. 1 and 2, the length of arms 202 and 204 suitably ranges from 1 to 3.5 inches, preferably 1.5 to 2 inches, and in the preferred embodiment, 1.5 inches. Arms 202 and 204 are preferably (but not necessarily) of the same length. In an alternative embodiment, shown in FIG. 4, arms 202′ and 204′ (and cavity 210′ (not shown in FIG. 4) are rectangular in cross-section.

Connecting portion 206 couples arms 202 and 204, and provides a point of attachment for handle 104. Connecting portion 206 may be of similar cross-section to arms 202 and 204, or may be enlarged to accommodate an attachment or supporting structure for handle 104 and/or a portion of cavity 210 of a larger cross-section than in arms 202 and 204 (e.g., twice the diameter of the cavity in the respective arms) to facilitate uniform suction. The bottom surface of connecting portion 206 is preferably coplanar with the bottom surface of arms 202 and 204.

Apertures 208 provide fluid communication between the suction system and fluids in the vicinity of incision 118, e.g., between interior cavity 210 and the exterior of base 102 in the vicinity of incision 118. As previously noted, in the embodiment of FIGS. 1 and 2, apertures 208 are disposed in the sidewalls of arms 202 and 204 (and connecting portion 206) facing nominal incision line 116. As shown, apertures 208 are evenly spaced along the sidewalls of arms 202 and 204 (and connecting portion 206) at predetermined intervals, in single or multiple rows. For example, apertures 208 are suitably arrayed in from 1 to 4 rows, and preferably in two or three rows. Where multiple rows are employed, adjacent rows may be offset. The number and diameter of apertures 208 are chosen such that apertures 208 are not overly susceptible to clogging, while at the same time providing the requisite suction and potential fluid flow rate. The diameter suitably ranges from 0.0625 to 0.125 inches, and preferably between 0.09375 to 0.15625 inches. The spacing interval between apertures 208 suitably ranges from 50 to 100 percent of the diameter of the apertures. In the preferred embodiment, 3 offset rows of 0.15625 inch diameter apertures spaced in intervals of 0.15625 inches are employed. The lowest row is preferably disposed on the lower curve of the arm sidewall touching or nearly touching the plane of the incision.

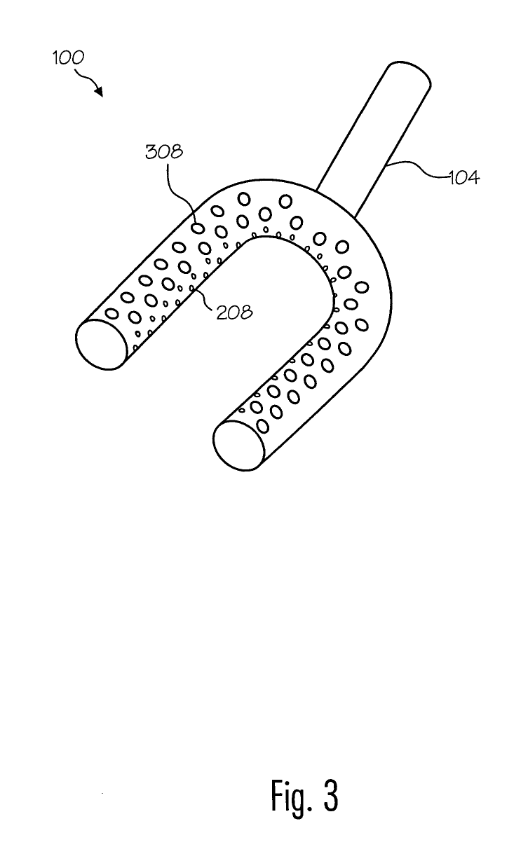

In certain procedures, suction is also applied after an incision has been closed (e.g., sewn shut). For example, in a cesarean section procedure, after the lower part of the uterus has been sewn shut, is often desirable to apply suction to the back portion of the uterus prior to closing the abdomen. Referring briefly to FIG. 3, if desired, additional apertures 308 (suitably of slightly (e.g., 50%) larger diameter than apertures 208) may be provided through the bottom surface of base 102 to facilitate such functionality. Apertures 308 would be used to suction off large volumes of blood and fluids when little or no pressure is being applied to occlude blood vessels in the vicinity of incision. When pressure is applied to occlude the blood vessels in the vicinity of incision, underlying tissue would close bottom-surface apertures 308, so that all suction is applied through sidewall apertures 208.

Handle 104 provides a mechanism for holding device 100, inserting base 102 into or onto the operating field, positioning base 102 relative to nominal line 116 along which incision 118 is to be made, e.g., lower part of the uterus, and exerting pressure on arms 202 and 204 to occlude the blood vessels in the vicinity of incision 118. In addition, in the embodiment of FIGS. 1 and 2, passageway 212 in handle 104 constitutes part of the suction system. In such embodiment, handle 104 is tubular (i.e., of annular cross-section) with predetermined outer diameter and inner diameter (defining passageway 212). The outer and inner diameters are chosen to provide sufficient strength to accommodate exerting the requisite pressure on arms 202 and 204, while at the same time accommodating the required level of suction and fluid flow rates. The diameter of passageway 212 is suitably a predetermined multiple of the diameter of conduit 210 in arms 202 and 204, e.g., ranging from 1 to 2 times the diameter of conduit 210. In the preferred embodiment, handle 104 has an outer diameter that suitably ranges from 0.25 to 0.625 inches, and preferably 0.375 to 0.5 inches. In the preferred embodiment, handle 104 is tubular, formed of plastic or metal, and has an outer diameter of 0.375 inches and inner diameter of 0.25 inches.

Handle 104, may be fixedly or removably attached to base 102. Any suitable mechanism, can be utilized to secure a removable handle 104 to base 102. For example, handle 104 can be closely received by an aperture in (or by an extension of) base 102, engaging the aperture in a friction fit. In the embodiment of FIG. 2, base 102 includes a threaded aperture 216 (communicating with cavity 210) and the proximal end 218 of handle 104 is threaded; aperture 216 is adapted to threadedly receive proximal end 218, although any method of connection may be used. The length of handle 104 is preferably sufficient to enable positioning base 102 relative to nominal line 116 along which incision 118 is to be made, e.g., lower part of the uterus, without obscuring the surgeon's view. The length of handle 104 is suitably about 8 to 12 inches, preferably 8 to 10 inches, and the most preferred embodiment is 9 inches.

In the preferred embodiment, handle 104 is disposed relative to base 102 so that it facilitates exerting appropriate pressure on the underlying tissue to occlude the blood vessels in the vicinity of incision 118 without obscuring the surgeon's view of the area in which incision 118 is to be made. However, if a handle is used, it could simply be of any shape and size suitable to be held by a surgeon; it need not be used to apply pressure. Handle 104 suitably attaches to base 102 at a point equidistant to arms 202 and 204 and suitably extends outwardly from base 102 in a plane along the centerline of base 102 that is perpendicular to the plane defined by the center lines of arms 202 and 204. Handle 104 is at a predetermined angle a (FIGS. 1 and 4) relative to the plane defined by the centerlines of arms 202 and 204. Predetermined angle a is suitably in the range of about 90 to 140 degrees, preferably 100 to 130 degrees, most preferably 110 to 120 degrees, and in the preferred embodiment is approximately 120 degrees.

Suctioning device 100 may be made from any autoclaveable material such as, for example, stainless steel. Alternatively, device 100 may be disposable, formed of plastic material with sufficient structural strength. For example, suction device 100 may be formed as an integral unit (or one or both of base 102 and a removable handle 104) by, e.g., conventional injection molding of plastic.

The foregoing is a description of preferred exemplary embodiments and best mode of the invention known to the applicant at the time of filing the application. The invention is not limited to the specific forms shown. For example, modifications may be made in the design and arrangement of the elements within the scope of the invention, as expressed in the appended claims.

1. A device for the removal of fluids during a surgical procedure, the device adapted for cooperation with a suction source and comprising:
(a) a base including a first arm, a second arm and an interior cavity extending into each of the arms, the interior cavity being adapted to have suction applied thereto;
(b) the first arm and the second arm being disposed to extend along opposite sides of a surgical incision line; and
(c) at least one arm including a plurality of apertures in fluid communication with the interior cavity, such that, responsive to a suction applied to the interior cavity, fluids in the vicinity of any of the plurality of apertures tend to be drawn into the interior cavity.

2. The device of claim 1 wherein when at least one of the arms is touching body tissue, the at least one arm tends to compress the tissue and occlude blood vessels on either side of the incision line in response to pressure applied to the at least one arm.

3. The device of claim 1 wherein each arm includes a plurality of apertures.

4. The device of claim 1 further including a handle.

5. The device of claim 4 wherein the handle extends from the base.

6. The device of claim 5 wherein the handle is removably attached to the base.

7. The device of claim 4 wherein the handle includes a passageway in the interior thereof and a fitting adapted for connection to a suction hose, the passageway communicating with the interior cavity.

8. The device of claim 2 that further includes a handle wherein the handle extends from the base at an angle chosen such that application of force to the handle causes the arms to compress the tissue and occlude the blood vessels on either side of the incision.

9. The device of claim 1 that further includes at least one aperture larger than any of the plurality of apertures.

10. The device of claim 1 wherein each of the arms has an annular cross section.

11. The device of claim 1 wherein each of the arms has a rectangular cross-section.

12. The device of claim 1 wherein the arms are parallel.

13. A device for the removal of fluids during a surgical procedure, the device comprising:
(a) a base including a first arm, a second arm and a connecting portion connecting the arms, there being a space between the first arm and the second arm;
(b) the base including an interior cavity extending from the connecting portion into each of the arms;
(c) the first arm having a first surface and the second arm having a second surface;
(d) the first surface including a plurality of apertures providing fluid communication between the interior cavity and the space between the arms; and
(e) the second surface including a plurality of apertures providing fluid communication between the interior cavity and the space between the arms; and
(f) a handle extending from the base.

14. The device of claim 13 wherein the handle includes a passageway for providing fluid communication between the interior cavity and a port, the port being adapted for cooperation with a suction source.

15. The device of claim 13 wherein the first arm has a longitudinal axis and the second arm has a longitudinal axis, each of the axes being located in a common plane.

16. The device of claim 15 wherein the longitudinal axes of the arms are substantially parallel.

17. The device of claim 15 wherein the longitudinal axes of the arms are substantially parallel to the first surface and the second surface.

18. The device of claim 13 wherein the handle extends from the base at an angle transverse to such plane.

19. The device of claim 18 wherein the angle is between 90 and 140 degrees.

20. The device of claim 13 wherein the connecting portion includes a third surface disposed between the first surface and the second surface, the third surface including a plurality of apertures providing fluid communication between the interior cavity and the space between the arms.

21. The device of claim 13 wherein the handle extends from the connecting portion.

22. The device of claim 13 wherein the first arm includes a fourth surface transverse to the first surface and the second arm includes a fifth surface transverse to the second surface, the fourth and fifth surfaces defining a plane; and the handle extends from the base at an angle transverse to such plane.

23. The device of claim 22 wherein the angle is between 90 and 140 degrees.

24. The device of claim 13 wherein the first arm and second arm are each circular in cross-section

25. The device of claim 14 wherein the handle conduit is longitudinally disposed, and the handle comprises a distal end comprising the port.

26. The device of claim 13 wherein the handle is removably attached to the base.

27. The device of claim 13 wherein the space between the arms is in the range of 0.25 to 0.75 inches.

28. The device of claim 27 wherein the space is in the range of 0.25 to 0.375 inches.
